(19) United States
(12) Patent Application Publication
(10) Pub. No.: US 2025/0352198 A1
(43) Pub. Date: Nov. 20, 2025

(54) SELF-STOPPING TISSUE ANCHORS

(71) Applicant: Edwards Lifesciences Innovation (Israel) Ltd., Caesarea (IL)

(72) Inventors: Haim Brauon, Beit Dagan (IL); Yuval Kasher, Kfar Shmuel (IL)

(21) Appl. No.: 19/290,125

(22) Filed: Aug. 4, 2025

Related U.S. Application Data
(63) Continuation of application No. 18/061,930, filed on Dec. 5, 2022, now Pat. No. 12,396,718.
Application No. 18/061,930 is a continuation of application No. PCT/IB2021/053314, filed on Apr. 22, 2021.
(60) Provisional application No. 63/041,423, filed on Jun. 19, 2020.

(51) Int. Cl. A61B 17/04

(52) U.S. Cl. CPC A61B 17/0401; A61B 2017/0443; A61B 2017/0464

(57) ABSTRACT

Using a driver, a tissue anchor is advanced to a tissue of a subject. The tissue anchor includes an anchoring portion, an anchor head fixedly coupled to the anchoring portion, and a driver interface. The anchoring portion is screwed distally into the tissue by, while the driver is engaged with the driver interface, applying torque, via the driver, to the driver interface such that the anchor head and the anchoring portion rotate together about a longitudinal axis of the tissue anchor. Continued screwing of the anchoring portion distally into the tissue is performed by continuing to apply the torque at least until the driver interface becomes rotatable relative to the anchor head. Subsequently, the driver is disengaged from the driver interface and removed from the subject, while leaving the tissue anchor anchored to the tissue. Other embodiments are also described.

### CROSS-REFERENCE TO RELATED APPLICATIONS

The present application is a continuation of a U.S. patent application Ser. No. 18/061,930, filed Dec. 5, 2022, which is a continuation of International Patent Application No. PCT/IB2021/053314, filed Apr. 22, 2021, and which claims the benefit of U.S. Patent Application No. 63/041,423, filed Jun. 19, 2020, the entire disclosures all of which are incorporated by reference for all purposes.

### BACKGROUND

Tissue anchors are useable in a range of medical applications (e.g., for fastening an implant to tissue). Screwable tissue anchors can be configured to translate torque into distal motion, whereby the anchor (e.g., an anchoring portion thereof) is screwed into tissue.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure can be included in the examples summarized here.

Some applications herein are directed to tissue anchors that facilitate controlled anchoring of the tissue anchors into tissue of a subject, such as cardiac tissue. That is, the tissue anchors themselves include features that facilitate such control. For some applications, additional apparatus or system components(s), such as an anchor driver, is provided for screwing an anchoring portion of the tissue anchor into the tissue.

For some applications, a tissue anchor limits a magnitude of torque which can be used to screw the anchor into tissue. For some applications, a tissue anchor limits a depth to which the tissue anchor can be screwed into tissue.

Some tissue anchors described herein can comprise an anchoring portion, and a crown coupled thereto. The crown can include an anchor head, a driver interface, and a socket. The anchor head can be fixedly coupled to the anchoring portion. The socket can be fixedly coupled to the driver interface and can further be shaped to receive the anchor head. In a first state the anchor head is seated snugly within the socket, such that torque applied to the driver interface is transferred to the anchoring portion, thereby facilitating screwing of the anchoring portion into the tissue. Screwing of the anchoring portion into the tissue pulls the anchor head distally out of the socket, thereby transitioning the anchor into a second state in which torque applied to the driver interface rotates the socket relative to the anchor head and the anchoring portion, e.g., such that the torque is no longer transferred to the anchoring portion, e.g., such that further screwing of the anchoring portion into the tissue is not possible.

Some tissue anchors described herein can comprise a crown including an anchor head that is fixedly coupled to the anchoring portion. The crown can define a driver interface configured to be engaged by a driver. The crown can also define a tissue-facing surface, such that screwing the anchoring portion into the tissue moves the tissue-facing surface distally toward the tissue.

For some applications, the crown can comprise a socket that can be fixedly coupled to the driver interface and is shaped to receive the anchor head. For some such applications, the tissue anchor can have (i) a torque-transfer state in which the anchor head is seated within the socket, such that torque applied by the driver to the driver interface rotates the socket, the anchor head, and the anchoring portion, thereby screwing the anchoring portion into the tissue, and (ii) a non-torque-transfer state in which the anchor head is disposed distally from the socket, such that applied torque is not transferred from the interface to the anchor head and the anchoring portion (or is at least significantly reduced).

For such applications, contact between the tissue-facing surface and the tissue can increase resistance against further distal movement of the tissue-facing surface. At this stage, further screwing of the anchor can pull the anchor head distally out of the socket (e.g., towards the tissue-facing surface), such that the tissue anchor transitions from the torque-transfer state to the non-torque-transfer state, thereby limiting the distal force applied by the tissue-facing surface to the tissue.

For some such applications, the tissue anchor can further comprise a spring disposed between the anchor head and the tissue-facing surface. Pulling the anchor head distally out of the socket and towards the tissue-facing surface can compress the spring between the anchor head and the tissue-facing surface, facilitating continued screwing of the anchoring portion into the tissue while the tissue anchor transitions from the torque-transfer state to the non-torque-transfer state.

For some applications, the crown can comprise a slip clutch that transfers torque from the driver interface to the anchor head, while limiting the transferred torque to not exceed a torque threshold.

For some applications in which the crown comprises a slip clutch, the slip clutch can transfer torque from the driver interface to the anchor head via a cantilever pin that revolves, with the anchor head, around the longitudinal axis of the anchor, while a torque-applying portion of the pin remains in contact with a noncircular lateral surface of the anchor head. However, when the applied torque exceeds the torque threshold, the torque-applying portion of the pin deflects away from the anchor head (e.g., due to being pushed laterally outward by the geometry of the anchor head), such that the driver interface and the pin can rotate with respect to the anchor head and the anchoring portion, thereby limiting the transferred torque.

For some such applications, deflection of the torque-applying portion of the pin away from the anchor head is dependent upon whether the applied torque is forward torque or reverse torque. For example, application of forward torque exceeding the torque threshold can cause the pin to deflect. However, application of reverse torque exceeding the torque threshold may not cause the pin to deflect. For example, the pin may revolve in a reverse rotational direction while the pin remains in contact with the anchor head, causing the anchoring portion to unscrew from the tissue. It is hypothesized that, in this way, the anchor can limit torque for screwing of the anchor into the tissue, while reliably allowing sufficient torque for unscrewing of the anchor from the tissue.

For some applications in which the crown comprises a slip clutch, the anchor head comprises a gear shaped to define a lateral surface and a notch. For such applications, the slip clutch transfers torque to the gear via a cantilever pin that revolves, with the anchor head, around the longitudinal axis of the anchor, while a torque-applying portion of the pin remains in contact with the gear. However, when the applied torque exceeds the torque threshold, the torque-applying portion of the pin deflects away from the gear (e.g., due to being pushed laterally outward by the geometry of the anchor head), such that the driver interface and the pin rotate with respect to the gear and the anchoring portion, thereby limiting the transferred torque.

For some such applications, deflection of the torque-applying portion of the pin away from the gear is dependent upon whether the torque is applied to the interface is forward torque or reverse torque. For example, application of forward torque exceeding the torque threshold can cause the pin to deflect away from the gear. However, application of reverse torque exceeding the torque threshold may not cause the pin to deflect. Instead, the pin can revolve in a reverse direction until an end-portion of the pin is latched into the notch defined by the gear. In this way, application of reverse torque causes the gear and the anchoring portion to rotate with the driver interface, causing the anchoring portion to unscrew from the tissue. It is hypothesized that, in this way, the anchor can limit torque for screwing of the anchor into the tissue, while reliably allowing sufficient torque for unscrewing of the anchor from the tissue.

There is therefore provided, in accordance with some applications, a system and/or apparatus for use with tissue of a subject, the system/apparatus including a driver and a tissue anchor, the tissue anchor including an anchoring portion configured to be screwed distally into the tissue by being rotated about a longitudinal axis of the anchor and a crown, coupled to a proximal portion of the anchoring portion, defining a tissue-facing surface.

In some applications, the tissue anchor and/or crown of the tissue anchor includes an anchor head fixedly coupled to the anchoring portion, such that screwing the anchoring portion into the tissue moves the anchor head distally along the longitudinal axis toward the tissue and/or a driver interface, configured to be engaged by the driver.

In some applications, the system/apparatus includes a socket, fixedly coupled to the driver interface, and shaped to receive the anchor head within the socket, the tissue-facing surface facing distally away from the socket.

In some applications, the system/apparatus or tissue anchor thereof has a first state in which the anchor head is seated snugly within the socket, such that torque applied by the driver to the driver interface rotates the socket, the anchor head, and the anchoring portion, thereby facilitating screwing of the anchoring portion into the tissue.

In some applications, the system/apparatus or tissue anchor thereof has a second state in which the anchor head is disposed distally from the socket, such that torque applied by the driver to the driver interface rotates the socket relative to the anchor head and the anchoring portion.

In some applications, the tissue anchor is configured to transition from the first state to the second state, responsively to the anchoring portion having been screwed into the tissue sufficiently deep such that the tissue resists further distal movement of the tissue-facing surface while the screwing of the anchoring portion into the tissue pulls the anchor head distally out of the socket.

In some applications, the driver interface defines a floor that separates the driver from the anchor head while the driver interface is engaged by the driver.

In some applications, the anchor head is shaped such that a transverse cross-section of the anchor head defines a non-circular profile. In some applications, the anchor head is shaped such that the transverse cross-section of the anchor head defines a plurality of lateral surfaces. In some applications, the anchor head is shaped such that the transverse cross-section of the anchor head defines a polygon. In some applications, the anchor head is shaped such that the transverse cross-section of the anchor head defines a square. In some applications, the anchor head is shaped such that the transverse cross-section of the anchor head defines a hexagon.

In some applications, the tissue is tissue of a heart of the subject, and the tissue anchor is transluminally advanceable to the heart.

In some applications, the driver includes a flexible shaft and a driver head at a distal end of the shaft, such that the anchor driver is transluminally advanceable to the heart.

In some applications, the crown includes a casing, the casing dimensioned to define: the driver interface, the socket, the tissue-facing surface, and a free zone disposed between the socket and the tissue-facing surface, and while the anchor is in the second state, the anchor head is disposed within the free zone.

In some applications, the anchor head is configured to rotate with respect to the socket while the anchor head is disposed in the free zone.

In some applications, the driver includes a driver head, the driver head shaped to define a shoulder, the shoulder: positioned on a side of the driver head, and dimensioned such that, while the driver interface is engaged by the driver head, the shoulder contacts a proximal surface of the casing.

In some applications, the system/apparatus includes a spring disposed within the casing, between the anchor head and the tissue-facing surface, and the anchor is configured such that while the anchor transitions from the first state to the second state: screwing the anchoring portion into the tissue pulls the anchor head distally out of the socket, compressing the spring.

In some applications, the anchor is configured such that while the anchor transitions from the first state to the second state, screwing the anchoring portion into the tissue pulls the anchor head distally out of the socket, compressing the spring and pressing the tissue-facing surface against the tissue.

In some applications, the anchor is configured such that while the anchor transitions from the first state to the second state, screwing the anchoring portion into the tissue pulls the anchor head distally out of the socket, compressing the spring while the anchor head is: partially disposed within the socket, and partially disposed within the free zone.

The system and/or apparatus can further comprise an implant, and the tissue anchor can be configured to secure the implant to the tissue. In some applications, the implant comprises a tether or contraction member. In some applications, the tissue anchor is configured to secure the tether or contraction member to the tissue. In some applications, the tissue anchor is configured to secure the tether or contraction member to the tissue such that applying tension to the tether or contraction member changes a shape and/or size of the tissue.

There is further provided, in accordance with some applications, a system and/or apparatus including: a driver, including a shaft and a driver head at a distal end of the shaft; and a tissue anchor. The tissue anchor includes an anchoring portion configured to be screwed distally into the tissue by being rotated about a longitudinal axis of the anchor.

In some applications, the tissue anchor includes a crown, coupled to a proximal portion of the anchoring portion.

In some applications, the tissue anchor and/or crown includes an anchor head fixedly coupled to the anchoring portion, such that rotation of the anchor head rotates the anchoring portion about the longitudinal axis.

In some applications, the tissue anchor, crown, and/or anchor head includes a driver interface, configured to be engaged by the driver head and rotated by the driver.

In some applications, the tissue anchor, crown, and/or anchor head includes a slip clutch. In some implementations, the slip clutch is coupled to the driver interface and/or to the anchor head. In some implementations, the slip clutch is configured to (i) transfer, to the anchor head, torque applied to the driver interface, up to a torque threshold, and to (ii) slip in response to torque greater than the torque threshold applied to the driver interface, thereby limiting torque transferred to the anchor head to not exceed the torque threshold.

In some applications, the anchor head is shaped to define a non-circular lateral surface, the slip clutch includes a cantilever pin, a portion of the cantilever pin fixedly coupled to the driver interface. In some implementations, the slip clutch is configured: (i) to transfer torque from the driver interface to the anchor head by revolving about the longitudinal axis in response to the driver interface rotating while a torque-applying portion of the pin is in contact with the non-circular lateral surface of the anchor head, and (ii) to slip by the pin being deflected away from the longitudinal axis by the anchor head.

In some applications, the slip clutch includes a cantilever pin, a portion of the cantilever pin fixedly coupled to the driver interface, and the slip clutch is configured to slip in response to torque greater than the torque threshold being applied to the driver interface, by the pin being deflected away from the longitudinal axis by the anchor head.

In some applications, the tissue is tissue of a heart of a subject, and the tissue anchor is transluminally advanceable to the heart.

In some applications, the driver includes a flexible shaft and a driver head at a distal end of the shaft, such that the anchor driver is transluminally advanceable to the heart.

In some applications, the slip clutch is configured to selectively rotationally couple the driver interface to the anchor head, such that: (i) in response to application, to the driver interface, of torque in a first rotational direction and at a first magnitude that does not exceed the torque threshold, the anchor head and the anchoring portion rotate with the driver interface in the first rotational direction; in response to application, to the driver interface, of torque in the first rotational direction and at a second magnitude that exceeds the torque threshold, the slip clutch slips such that the driver interface rotates with respect to the anchor head and the anchoring portion; and in response to application, to the driver interface, of torque in a second rotational direction and at the second magnitude, the anchor head and the anchoring portion rotate with the driver interface in the second rotational direction, the second rotational direction being opposite to the first rotational direction.

In some applications, the torque threshold is a first torque threshold, and the tissue anchor is configured such that application, to the driver interface, of torque in the second rotational direction and at a third magnitude exceeding a second torque threshold that is greater than the first torque threshold causes the slip clutch to slip such that the driver interface rotates with respect to the anchor head and the anchoring portion.

In some applications, the anchoring portion is oriented with respect to the slip clutch such that: rotation of the anchor head and the anchoring portion in the first rotational direction facilitates screwing of the tissue anchor into the tissue, and rotation of the anchor head and the anchoring portion in the second rotational direction facilitates unscrewing of the tissue anchor from the tissue.

In some applications, the anchor head is shaped to define a non-circular lateral surface, the slip clutch includes a cantilever pin, a fixed portion of the cantilever pin fixedly coupled to the driver interface. In some implementations, the slip clutch is configured: to transfer torque from the driver interface to the anchor head by revolving about the longitudinal axis in response to the driver interface rotating while a torque-applying portion of the pin is in contact with the non-circular lateral surface of the anchor head, and to slip by the pin being deflected away from the longitudinal axis by the anchor head.

In some applications, the slip clutch is configured such that application of torque to the driver interface, in the first rotational direction and at the first magnitude, causes the anchor head and the anchoring portion to rotate with the driver interface, by the cantilever pin revolving about the longitudinal axis in the first rotational direction with the torque-applying portion ahead of the fixed portion.

In some applications, the slip clutch is configured such that application of torque to the driver interface, in the first rotational direction and at the first magnitude, causes the anchor head and the anchoring portion to rotate with the driver interface, by the cantilever pin revolving about the longitudinal axis in the first rotational direction with the torque-applying portion trailing the fixed portion.

In some applications, the slip clutch is configured such that: the torque-applying portion is a first torque-applying portion, and application of torque to the driver interface, in the first rotational direction and at the first magnitude, causes the anchor head and the anchoring portion to rotate with the driver interface, by the cantilever pin revolving about the longitudinal axis in the first rotational direction while the first torque-applying portion is in contact with the non-circular lateral surface of the anchor head. In some implementations, application of torque, to the driver interface, in the first rotational direction and at the second magnitude, causes the driver interface and the pin to rotate in the first rotational direction, with respect to the anchor head and the anchoring portion, by the pin being deflected away from the longitudinal axis by the anchor head. In some implementations, application of torque, to the driver interface, in the second rotational direction and at the second magnitude, causes the anchor head and the anchoring portion to rotate with the driver interface in the second rotational direction, by the cantilever pin revolving about the longitudinal axis in the second rotational direction while a second torque-applying portion of the pin is in contact with the anchor head.

In some applications, the slip clutch is configured such that: while torque is applied to the driver interface in the first rotational direction and at the first magnitude, the pin has a forward cantilever span between (i) the fixed portion of the pin, and (ii) the first torque-applying portion, and while torque is applied to the driver interface in the second rotational direction, the pin has a reverse cantilever span between (i) the fixed portion of the pin, and (ii) the second torque-applying portion, the forward cantilever span being longer than the reverse cantilever span.

In some applications, the first torque-applying portion is further than the second torque-applying portion from the fixed portion. In some applications, the second torque-applying portion is further than the first torque-applying portion from the fixed portion.

In some applications: the anchor head is shaped to define a notch, the pin defines a pawl, which serves as the second torque-applying portion, and the slip clutch is configured such that application of torque to the driver interface in the second rotational direction and at the second magnitude, causes the anchor head and the anchoring portion to rotate with the driver interface in the second rotational direction, by the cantilever pin revolving about the longitudinal axis in the second rotational direction while the pawl is latched into the notch defined by the anchor head.

In some applications, the slip clutch is configured such that application of torque to the driver interface in the second rotational direction and at the second magnitude, causes the driver interface and the pin to rotate in the second rotational direction, with respect to the anchor head and the anchoring portion, for not more than a quarter turn, before the pawl becomes latched into the notch defined by the anchor head.

The system and/or apparatus can further comprise an implant, and the tissue anchor can be configured to secure the implant to the tissue. In some applications, the implant comprises a tether or contraction member. In some applications, the tissue anchor is configured to secure the tether or contraction member to the tissue. In some applications, the tissue anchor is configured to secure the tether or contraction member to the tissue such that applying tension to the tether or contraction member changes a shape and/or size of the tissue.

There is further provided, in accordance with some applications, a method for use with a tissue of a subject includes advancing a tissue anchor to the tissue, the tissue anchor including an anchoring portion, anchor head, and a driver interface, and screwing the anchoring portion distally into the tissue by applying torque to the driver interface, such that the driver interface and the anchoring portion rotate together about a longitudinal axis of the anchor, continuing to screw the anchoring portion distally into the tissue, at least until the driver interface becomes rotatable relative to the anchor head.

In some applications, the tissue anchor includes a crown coupled to a proximal portion of the anchoring portion, and the crown can include the anchor head, which can be fixedly coupled to the anchoring portion. The driver interface can also be part of the crown in some implementations.

The method can further include engaging a driver (e.g., an anchor driver, etc.) with the driver interface. The method can further include using the driver to screw the anchoring portion distally into the tissue by applying torque to the driver interface, such that the driver interface, the anchor head, and the anchoring portion rotate together about a longitudinal axis of the anchor.

The method can further include, subsequently to screwing the anchor, disengaging the driver from the driver interface and removing the driver from the subject, while leaving the tissue anchor anchored to the tissue.

In some applications, the driver includes a driver head, the driver head shaped to define a shoulder, and screwing the anchoring portion distally into the tissue by applying torque to the driver interface includes screwing the anchoring portion distally into the tissue by applying torque to the driver interface while: the driver head is engaged with the driver interface, and the shoulder is in contact with a proximal surface of the crown.

In some applications, the driver interface defines a floor, and screwing the anchoring portion distally into the tissue by applying torque to the driver interface includes, using the driver head, contacting the floor.

In some applications, the tissue is tissue of a heart of the subject and advancing the tissue anchor to the tissue includes transluminally advancing the tissue anchor to the heart. In some implementations, the tissue is tissue of a mitral valve or a tricuspid valve of a heart.

In some applications, the driver includes a flexible shaft and a driver head at a distal end of the shaft, and the method includes, subsequently to advancing the tissue anchor to the tissue, using the flexible shaft, transluminally advancing the driver head to the driver interface.

In some applications, the crown is shaped to define: a socket fixedly coupled to the driver interface, and a tissue-facing surface facing distally away from the socket; and screwing the anchoring portion distally into the tissue includes screwing the anchoring portion distally into the tissue by applying torque to the driver interface while the anchor head is seated snugly within the socket, such that the driver interface, the socket, the anchor head, and the anchoring portion rotate about the longitudinal axis; and continuing to screw the anchoring portion distally into the tissue includes continuing to screw the anchoring portion distally into the tissue at least until: the tissue resists further distal movement of the tissue-facing surface, and the anchor head becomes pulled distally out of the socket, such that the socket becomes rotatable relative to the anchor head.

In some applications, the crown and/or anchor head is shaped to define a free zone between the socket and the tissue-facing surface, and continuing to screw the anchoring portion distally into the tissue includes continuing to screw the anchoring portion distally into the tissue at least until the anchor head becomes pulled distally into the free zone.

In some applications, continuing to screw the anchoring portion distally into the tissue includes continuing to screw the anchoring portion distally into the tissue at least until the anchor head becomes pulled entirely into the free zone.

In some applications, the tissue anchor includes a spring, the spring disposed between the anchor head and the tissue-facing surface, and continuing to screw the anchoring portion distally into the tissue includes continuing to screw the anchoring portion distally into the tissue at least until the anchor head compresses the spring.

In some applications, continuing to screw the anchoring portion distally into the tissue includes continuing to screw the anchoring portion distally into the tissue at least until the spring presses the tissue-facing surface against the tissue.

In some applications, the crown includes a slip clutch, the slip clutch being coupled to the driver interface and to the anchor head, screwing the anchoring portion distally into the tissue includes screwing the anchoring portion distally into the tissue by applying torque to the driver interface, such that torque is transferred from the driver interface, via the slip clutch, to the anchor head, and continuing to screw the anchoring portion distally into the tissue includes continuing to screw the anchoring portion distally into the tissue at least until, at a torque threshold, the slip clutch slips, such that the driver interface rotates with respect to the anchor head and the anchoring portion.

In some applications, the anchor head is shaped to define a non-circular lateral surface, the slip clutch includes a cantilever pin, a fixed portion of the cantilever pin being fixedly coupled to the driver interface, screwing the anchoring portion distally into the tissue includes screwing the anchoring portion distally into the tissue by applying torque to the driver interface, such that the driver interface and the pin rotate about the longitudinal axis in a first rotational direction, together with the anchor head and the anchoring portion, while a torque-applying portion of the pin presses against the lateral surface of the anchor head. In some implementations, continuing to screw the anchoring portion distally into the tissue includes continuing to screw the anchoring portion distally into the tissue at least until, at the torque threshold, the slip clutch slips by the anchor head deflecting the pin away from the longitudinal axis, such that the driver interface rotates with respect to the anchor head and the anchoring portion.

In some applications, the torque-applying portion of the pin is a first torque-applying portion of the pin, screwing the anchoring portion distally into the tissue includes screwing the anchoring portion distally into the tissue by applying torque to the driver interface, such that the driver interface and the pin rotate about the longitudinal axis in the first rotational direction, together with the anchor head and the anchoring portion, while the first torque-applying portion of the pin presses against the lateral surface of the anchor head. In some implementations, continuing to screw the anchoring portion distally into the tissue includes continuing to screw the anchoring portion distally into the tissue at least until, at the torque threshold, the slip clutch slips by the anchor head pushing against the first torque-applying portion and thereby deflecting the pin away from the longitudinal axis, such that the driver interface rotates with respect to the anchor head and the anchoring portion.

In some applications, the method includes unscrewing the anchoring portion proximally from the tissue, by applying torque in a second rotational direction to the driver interface, such that the anchor head and the anchoring portion rotate with the driver interface, in the second rotational direction while a second torque-applying portion of the pin presses against the anchor head.

In some applications, the anchor head is shaped to define a notch, the pin defines a pawl that serves as the second torque-applying portion, and unscrewing the anchoring portion proximally from the tissue includes unscrewing the anchoring portion proximally from the tissue, by applying torque, in the second rotational direction, to the driver interface, such that the anchor head and the anchoring portion rotate with the driver interface, in the second rotational direction while the pawl is latched into the notch.

In some applications, the methods herein further comprise anchoring an implant, tether and/or contraction member to the tissue. The tissue anchor can be configured to anchor or secure the implant, tether, and/or contraction member to the tissue. In some applications, the tissue anchor is used to anchor or secure the implant, tether, and/or contraction member to the tissue.

Anchoring or securing the implant, tether, and/or contraction member to the tissue can comprise screwing the anchoring portion distally into the tissue by applying torque to the driver interface, such that the driver interface and the anchoring portion rotate together about a longitudinal axis of the anchor, and continuing to screw the anchoring portion distally into the tissue, at least until the driver interface becomes rotatable relative to the anchor head.

In some applications, after anchoring or securing the implant, tether, and/or contraction member to the tissue, the method further comprises applying tension to the tether or contraction member to change a shape and/or size of the tissue (e.g., to change a shape and/or size of an annulus of a heart valve, etc.)

The above method(s) can be performed on a living animal or on a simulation, such as on a cadaver, cadaver heart, simulator (e.g., with the body parts, heart, tissue, etc. being simulated), etc.

The present invention will be more fully understood from the following detailed description of applications thereof, taken together with the drawings, in which:

### BRIEF DESCRIPTION OF THE DRAWINGS

FIGS. 1A-E, 2A-H, and 3A-H are schematic illustrations showing example tissue anchor systems and their use, in accordance with some applications;

FIGS. 4A-D, 5A-E and 6A-B are schematic illustrations showing an example tissue anchor system, in accordance with some applications; and

FIGS. 7A-C, 8A-E and 9A-C are schematic illustrations showing an example tissue anchor system, in accordance with some applications.

### DETAILED DESCRIPTION

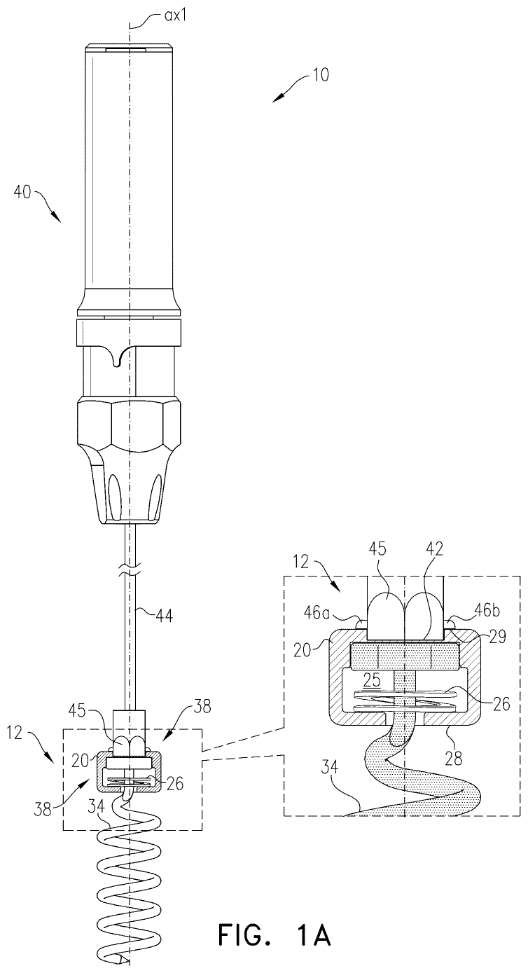

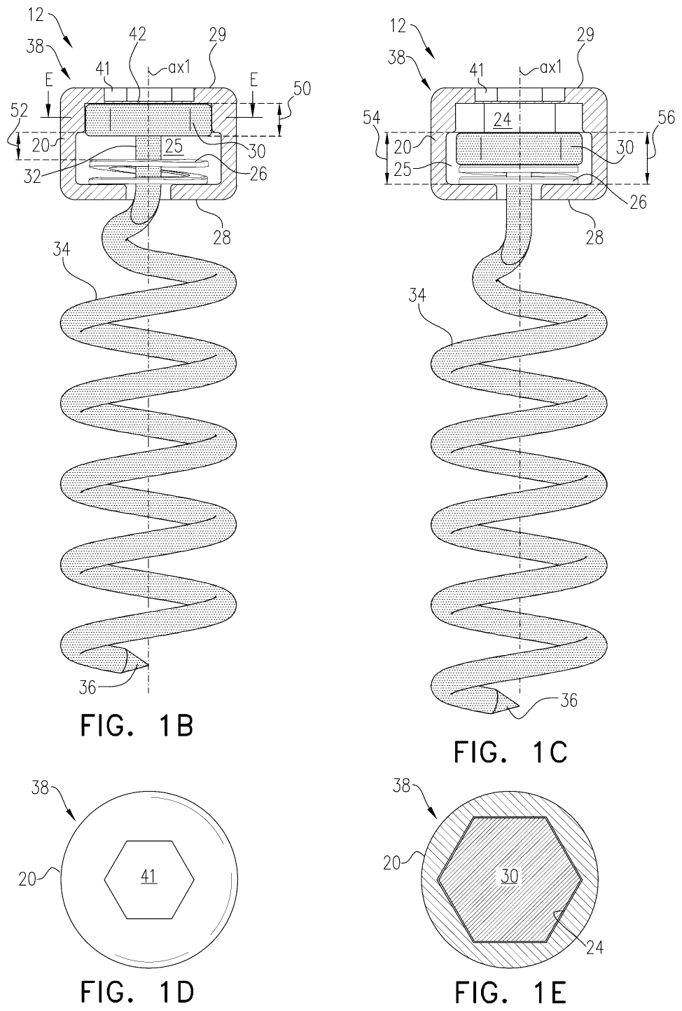

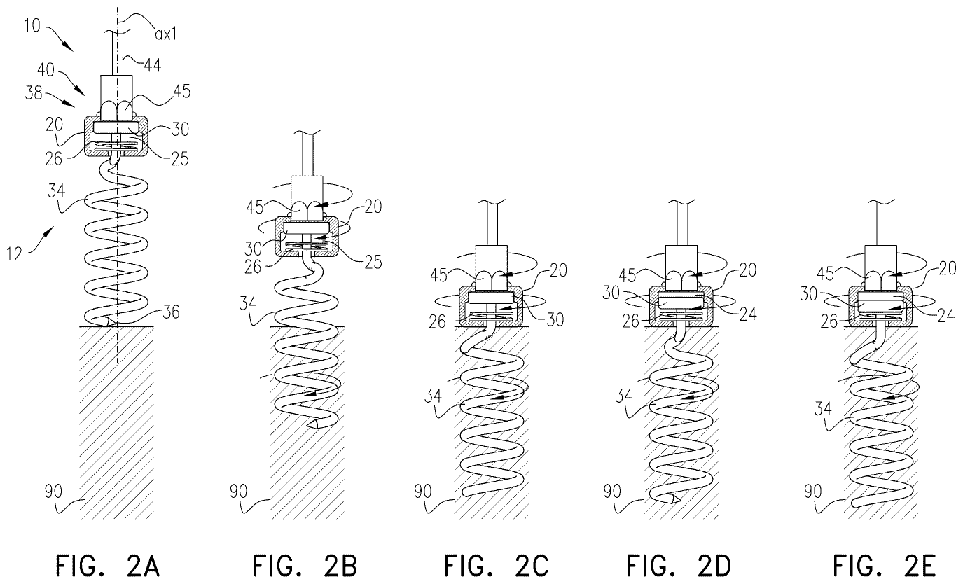

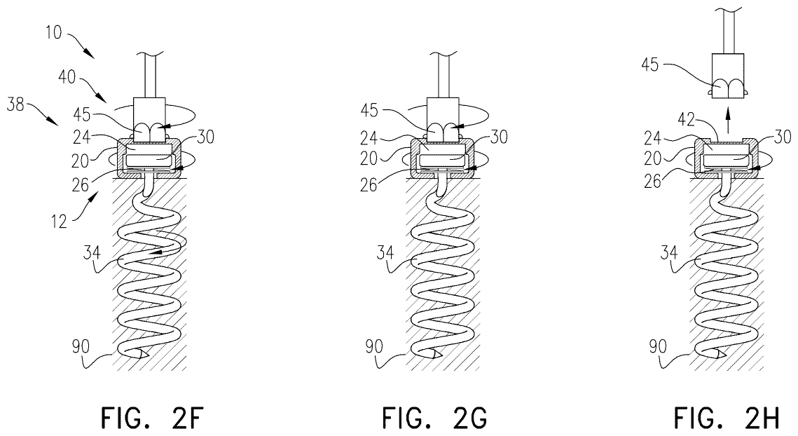

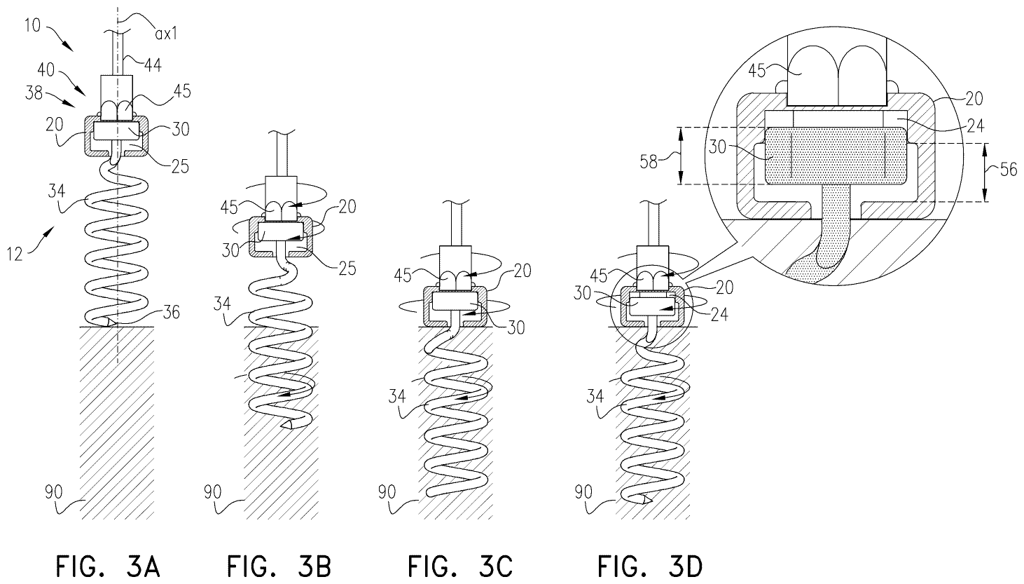

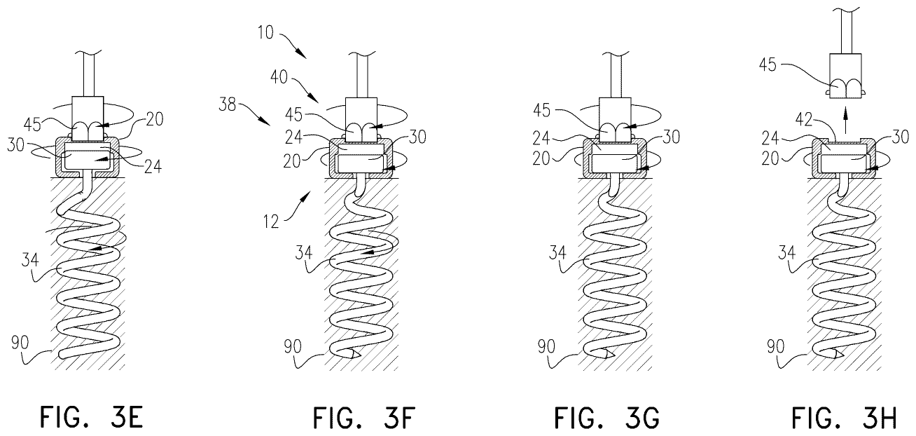

Reference is made to FIGS. 1A-E, 2A-H, and 3A-H, which are schematic illustrations showing use of an example tissue anchor system 10, in accordance with some applications.

System 10 comprises a tissue anchor 12, and an anchor driver 40. As shown, anchor 12 comprises an anchoring portion (i.e., a tissue-engaging element) 34 shaped to facilitate screwing of the anchoring portion into tissue 90 by being rotated about a longitudinal axis ax1 of anchor 12. For example, and as shown, anchoring portion 34 is shaped as a corkscrew having a distal tissue-piercing point 36. This is not meant to exclude other shapes which facilitate anchoring portion 34 being screwed into tissue 90. For example, anchoring portion can be shaped to define a threaded shank.

In some applications, and as shown, anchoring portion 34 is coupled, at a proximal portion of the anchoring portion, to a crown 38 that defines a tissue-facing surface 28. FIGS. 1A-C are side-views that show crown 38 in longitudinal cross-section (but anchoring portion 34 is shown whole). FIG. 1D is a top-view of anchor 12, i.e., looking down at crown 38. FIG. 1E is a transverse cross-section through crown 38, at the level indicated in FIG. 1B. As shown, crown 38 comprises an anchor head 30 and a socket 24 shaped to receive the anchor head, e.g., by the anchor head being reversibly seated within the socket. FIG. 1B shows anchor head 30 seated within socket 24, and FIG. 1C shows the anchor head having exited the socket. For some such applications, and as shown in cross-section in FIG. 1E, socket 24 and anchor head 30 are shaped complementarily to each other, such that the socket snugly receives the anchor head.

In some applications, and as shown, crown 38 comprises a driver interface 41 coupled to socket 24 such that the interface and the socket are rotationally fixed. Driver 40 is configured to engage the driver interface 41. For some applications, driver 40 is transluminally advanced to tissue 90 of a subject (e.g., tissue of a heart of the subject), e.g., while coupled to anchor 12. For some such applications, driver 40 having a shaft 44 (e.g., a flexible shaft) facilitates transluminal advancement of the driver to tissue 90.

In system 10, torque is transferred indirectly from driver 40 to anchor head 30, e.g., via driver interface 41 and socket 24. For example, driver 40 can comprise a driver head 45 at a distal end of shaft 44, that is reversibly seatable within driver interface 41 (FIG. 1A). For some applications, and as shown, driver 40 (e.g., driver head 45) does not contact anchor head 30 while driver head 45 is seated within driver interface 41. For example, anchor head 30 can be inaccessible to driver head 45 due to the anchor head being enclosed in a casing 20, e.g., as described hereinbelow.

In some applications, tissue-facing surface 28 faces distally away from socket 24. In some applications, tissue-facing surface 28 is axially fixed in relation to driver interface 41, such that screwing tissue anchor 12 into tissue 90 typically brings surface 28 closer to the tissue (e.g., brings the tissue-facing surface into contact with the tissue). For some applications, tissue-facing surface 28 is also rotationally fixed with respect to driver interface 41 (e.g., tissue-facing surface is fixedly attached to driver interface 41).

For some applications, and as shown, anchor 12 (e.g., crown 38 thereof) comprises casing 20 that comprises socket 24, interface 41 and surface 28. Casing 20 can be a unitary structure that is shaped to define socket 24, interface 41 and surface 28. Casing 20 can house anchor head 30, such that crown 38 comprises both the anchor head and the casing that houses the anchor head. Often for such applications, casing 20 is dimensioned to define a free zone 25 within which anchor head 30 is disposed while the anchor head is not seated in socket 24 (FIG. 1C). Free zone 25 can be disposed distally from socket 24 (i.e., closer than socket 24 to anchoring portion 34). Although socket 24 is configured to transfer torque from interface 41 to anchor head 30 while the anchor head is disposed in the socket (e.g., due to the snug fit therebetween), free zone 25 is configured to allow the socket to rotate with respect to the anchor head while the anchor head is disposed in the free zone and not in the socket (e.g., such that torque is not transferred from the socket to the anchor head).

For some applications, socket 24, interface 41, and surface 28 of casing 20 are rotationally fixed in relation to each other, such that rotation of one part of the casing rotates the entire casing. This is not meant to exclude applications in which tissue-facing surface 28, socket 24 and/or interface 41 are discrete elements.

As described hereinabove, system 10 can be configured such that anchor head 30 is inaccessible to driver 40 (e.g., to anchor head 45 thereof). For some applications, interface 41 (or another component of crown 38) defines a floor 42, which separates driver 40 from anchor head 30 while the driver is seated within the interface. Alternatively or additionally, driver 40 (e.g., driver head 45 thereof) is shaped to define one or more shoulders 46 (e.g., positioned laterally, as shown in FIG. 1A). Often for such applications, shoulders 46 are dimensioned such that, while driver interface 41 is engaged by driver 40 (e.g., while driver head 45 is seated within the interface), the shoulders contact a proximal surface 29 of casing 20. Anchor head 30 being inaccessible to driver 40 facilitates transfer of torque from driver 40 to driver interface 41, while reducing (e.g., eliminating) direct transfer of a pushing force from the driver to anchor head 30, e.g., restricting any transfer of the pushing force to be via casing 20.

In some applications, and as shown, anchor 12 is transitionable between a first state (e.g., a torque-transfer state, FIG. 1B) and a second state (e.g., a non-torque-transfer state, FIG. 1C). In some applications, and as shown, while anchor 12 is in the first state, anchor head 30 is seated within socket 24. In this way, torque applied by driver 40 to driver interface 41 rotates socket 24, anchor head 30, and anchoring portion 34. In some applications, and as described hereinbelow, the first state facilitates screwing anchoring portion 34 into tissue 90.

Further in some applications and as shown, while anchor 12 is in the second state, anchor head 30 is disposed outside of (e.g., distally from) socket 24, such that torque applied by driver 40 to driver interface 41 rotates socket 24 relative to anchor head 30 (and thereby anchoring portion 34), e.g., such that torque is not transferred from the driver to the anchor head and the anchoring portion. Transition of anchor 12 from the first state to the second state occurs in response to anchor head 30 being pulled distally out of socket 24 by screwing of the anchor into tissue 90, e.g., as described hereinbelow in reference to FIGS. 2A-H.

For some applications, and as shown, anchor 12 further comprises a compression spring 26 that can be disposed between anchor head 30 and tissue-facing surface 28 (e.g., within casing 20). The function of spring 26 is described in more detail hereinbelow.

FIGS. 2A-H show driver 40 being used to screw anchoring portion 34 of anchor 12 into tissue 90 while anchor head 30 moves distally along longitudinal axis ax1, pressing surface 28 against the tissue. FIGS. 3A-H show corresponding steps, but with a variant 12′ of anchor 12 that does not comprise compression spring 26. Henceforth, this variant of anchor 12 is referred to as anchor 12′.

FIG. 2A shows anchor 12 disposed against tissue 90, such that distal tissue-piercing point 36 contacts the tissue. Torque is then applied from driver head 45, via interface 41 and socket 24, to anchor 12, screwing anchoring portion 34 into tissue 90 (FIG. 2B). During this time, anchor 12 is in its torque-transfer state, and typically behaves similarly to a prior art tissue anchor of unitary construction. Thus, screwing of anchoring portion 34 into tissue 90 results in tissue-facing surface 28 moving distally along the longitudinal axis, until it contacts the tissue (FIG. 2C).

FIGS. 2D-F show further screwing of anchoring portion 34 into tissue 90, by continued rotation of driver head 45 and crown 38, despite anchoring portion 34 having already been screwed into tissue 90 sufficiently deep such that the tissue resists further distal movement of tissue-facing surface 28 and socket 24 (and often the entirety of casing 20). As shown in FIGS. 2D-F, this resistance contributes to the transition of the anchor from the first state to the second state, because once surface 28 has made contact with tissue 90 further screwing of anchoring portion 34 into the tissue progressively pulls anchor head 30 distally out of socket 24, which is inhibited by tissue 90 from advancing further distally. Since resistance from tissue 90 contributes to the transition of anchor 12 to the second state, torque applied to driver interface 41 is translated into distal motion of anchor head 30 relative to tissue-facing surface 28 (e.g., within casing 20), instead of being translated into application of a distal force by tissue-facing surface 28 to tissue 90. It is therefore hypothesized that triggering transition of anchor 12 from the first state to the second state, by tissue 90 resisting further distal movement of tissue-facing surface 28, may advantageously limit: (i) the distal force applied by the tissue-facing surface to the tissue while the anchor is screwed into the tissue, and/or (ii) a depth to which the tissue anchor can be screwed into the tissue.

In FIGS. 2D-E, anchor head 30 has moved further distally within casing 20, but has not yet completely exited socket 24 (e.g., the anchor head is partially disposed within the socket, and partially disposed within the free zone). Therefore, torque transfer to anchoring portion 34 is still possible. In FIG. 2F, anchor 12 has transitioned into its non-torque-transfer state, as anchor head 30 has been pulled completely out of socket 24 (i.e., into free zone 25), thereby rotationally disconnecting the socket from the anchor head. FIG. 2G illustrates that further rotation of driver head 45, rotates interface 41 and socket 24 (e.g., the entirety of casing 20), but does not result in further rotation of anchor head 30, or further screwing of anchoring portion 34 into the tissue. At this point in advancement of anchor 12 into tissue 90, torque applied by driver 40 to driver interface 41 rotates socket 24 relative to anchor head 30 and anchoring portion 34. It is therefore hypothesized that the transitioning of anchor 12 from the first state to the second state, in response to resistance from tissue 90 to tissue-facing surface 28, advantageously reduces a risk of overtightening or damaging the tissue contacted by tissue-facing surface 28.

At this point, the screwing of anchor 12 into tissue 90 is typically complete, and driver 40 can be removed (FIG. 2H).

As described briefly hereinabove, for some applications, and as shown, anchor 12 comprises compression spring 26 disposed within casing 20 (e.g., within free zone 25). For some such applications, spring 26 facilitates sustained screwing of anchor 12 into tissue 90 while the anchor transitions from the first state to the second state. Spring 26 can be disposed between anchor head 30 and tissue-facing surface 28. As anchor head 30 becomes progressively pulled out of from socket 24, and before the anchor head exits the socket entirely, the anchor head contacts spring 26 (FIG. 2E), such that further rotation of anchor head 30 begins to compress the spring, such that the spring presses tissue-facing surface 28 against tissue 90. It is hypothesized that, for some applications, spring 26 thereby advantageously increases reliability of anchor 12, by increasing a likelihood that tissue-facing surface 28 becomes pressed securely against tissue 90 before anchor 12 transitions into its non-torque-transfer state. To facilitate the described function of spring 26, while the spring is in a relaxed state (e.g., before anchor 12 has been introduced into the subject) an axial height 50 of anchor head 30 can be greater than an axial distance 52 between socket 24 (e.g., a distal end thereof) and spring 26. In the non-torque-transfer state of anchor 12, (e.g., once anchor 12 has been screwed into tissue 90), a combined axial height 54 of anchor head 30 and spring 26 can be similar to, but imperceptibly smaller than, an axial distance 56 between socket 24 (e.g., a distal end thereof) and a distal end of free zone 25. (Axial distance 56 can, for some applications, be considered the axial height of free zone 25.)

FIGS. 3A-H show the same sequence as FIGS. 2A-H, mutatis mutandis, but for the anchoring of anchor 12′. Anchor 12′ is typically as described for anchor 12, except that anchor 12′ lacks spring 26, and can be dimensioned differently in order to accommodate this lack of the spring. Anchor head 30 is typically dimensioned such that, upon the anchor head pressing tissue-facing surface 28 against tissue 90, the anchor head exits socket 24 (FIG. 3F), thereby transitioning anchor 12′ into its non-torque-transfer state (FIG. 3G).

For some applications, to confer reliability on anchor 12′, e.g., to reduce a likelihood that anchor 12′ transitions into its non-torque-transfer state before its tissue-facing surface 28 becomes pressed securely against tissue 90, an axial height 58 of anchor head 30 can be similar to, but imperceptibly smaller than, axial distance 56.

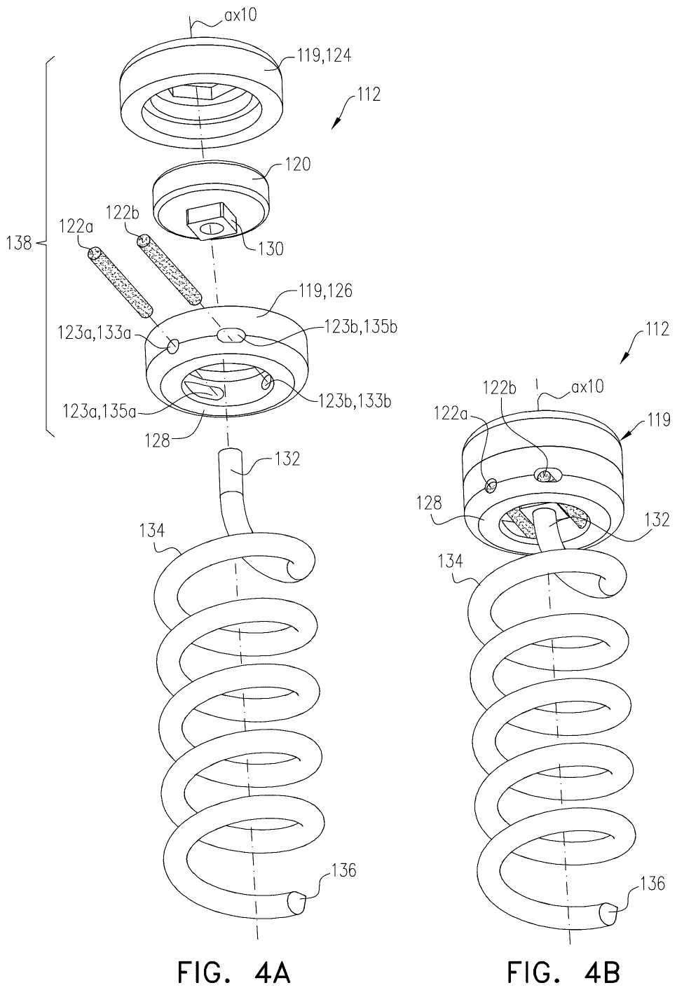

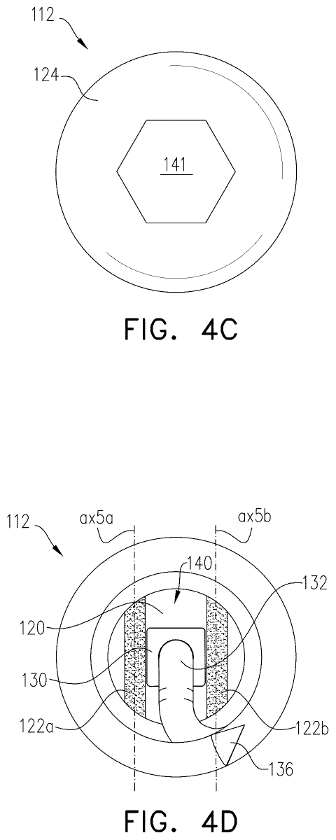

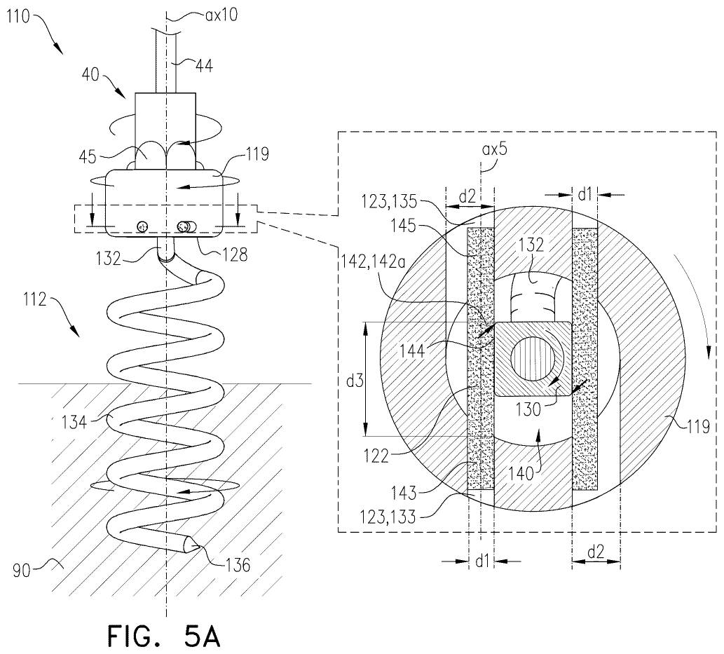

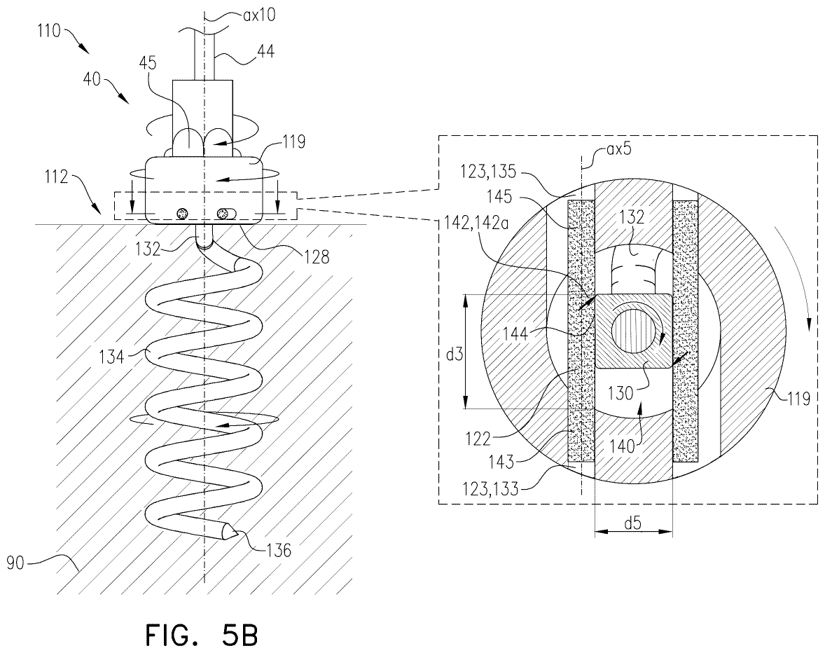

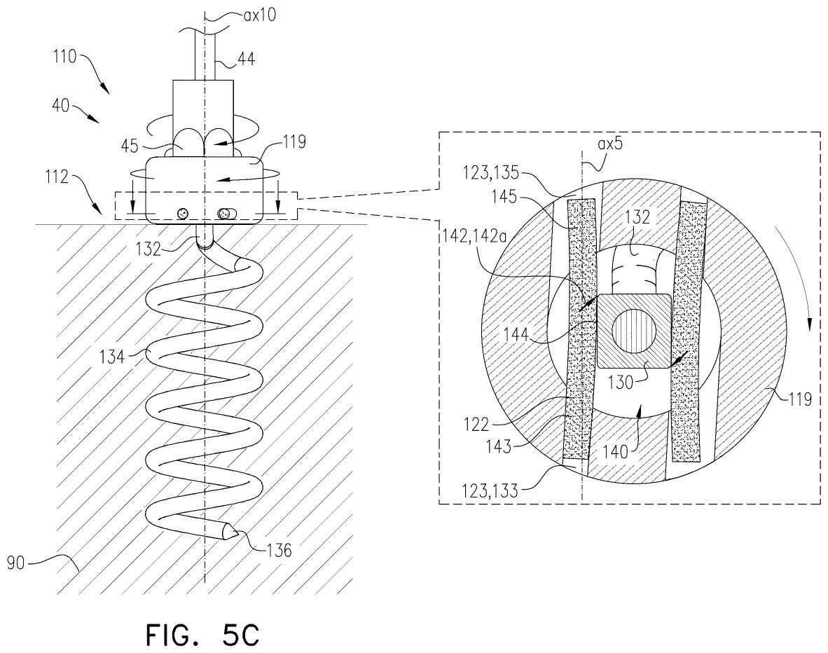

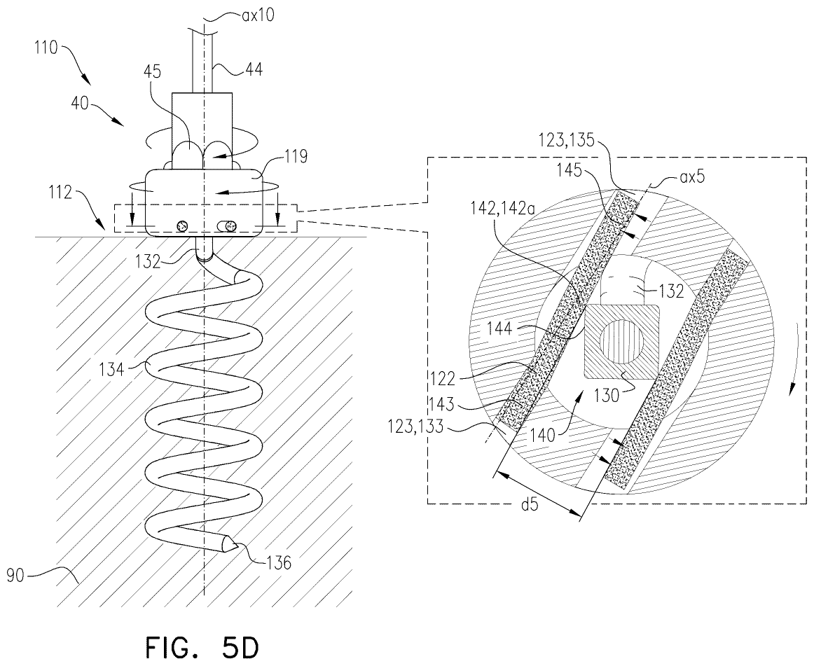

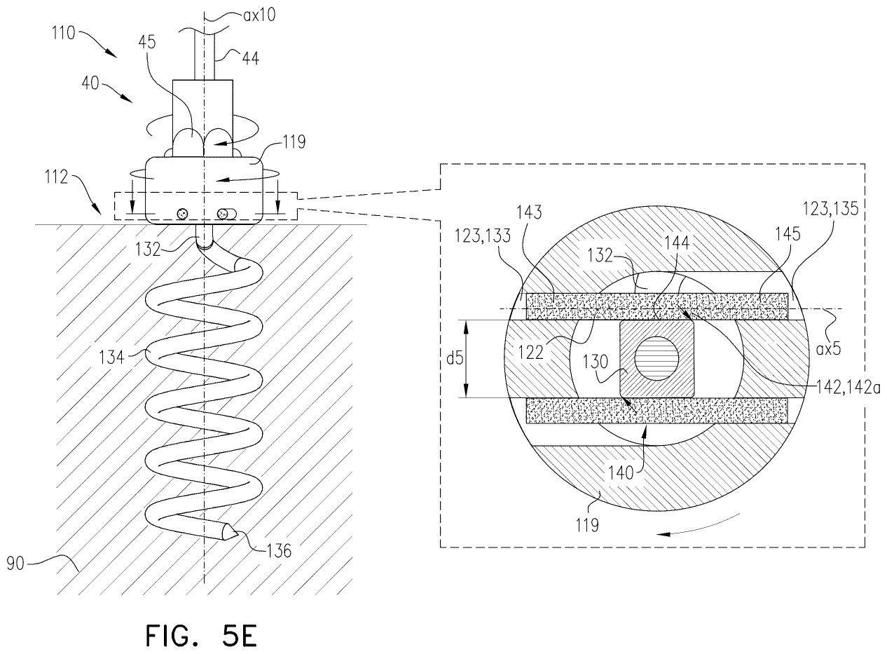

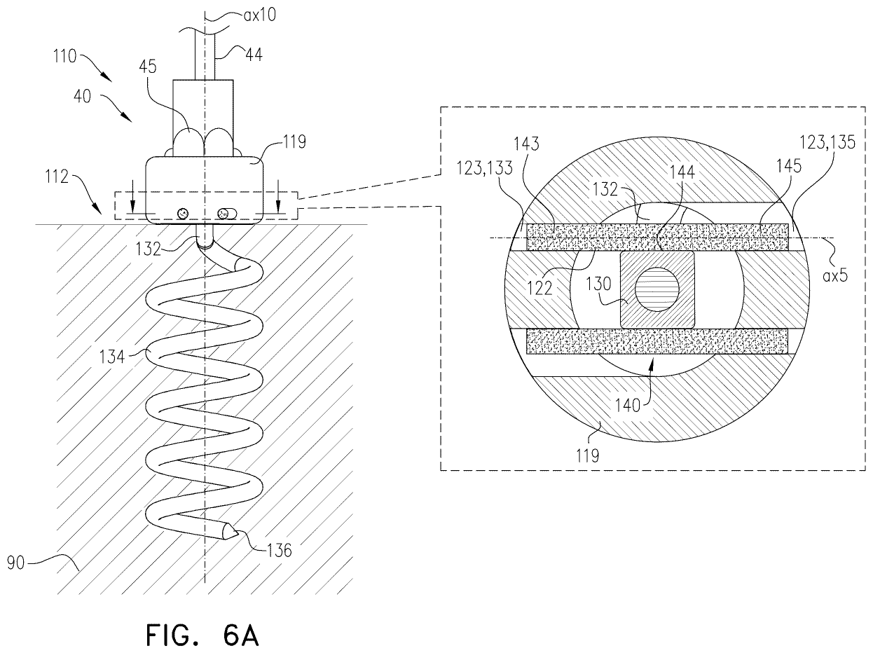

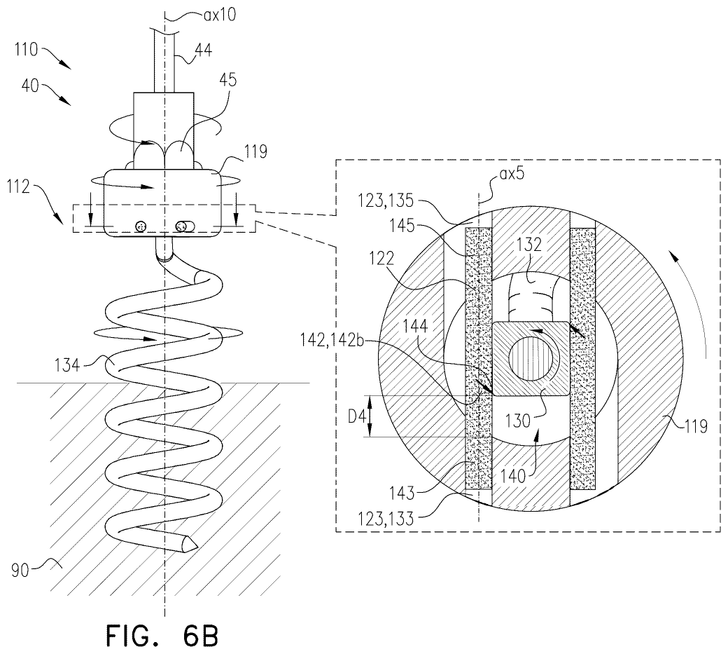

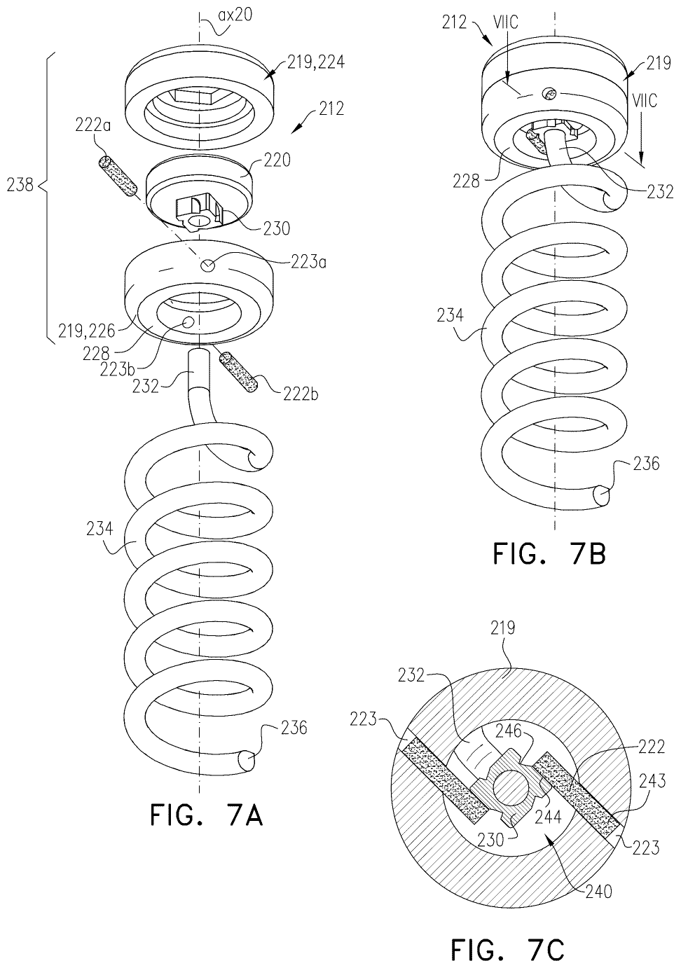

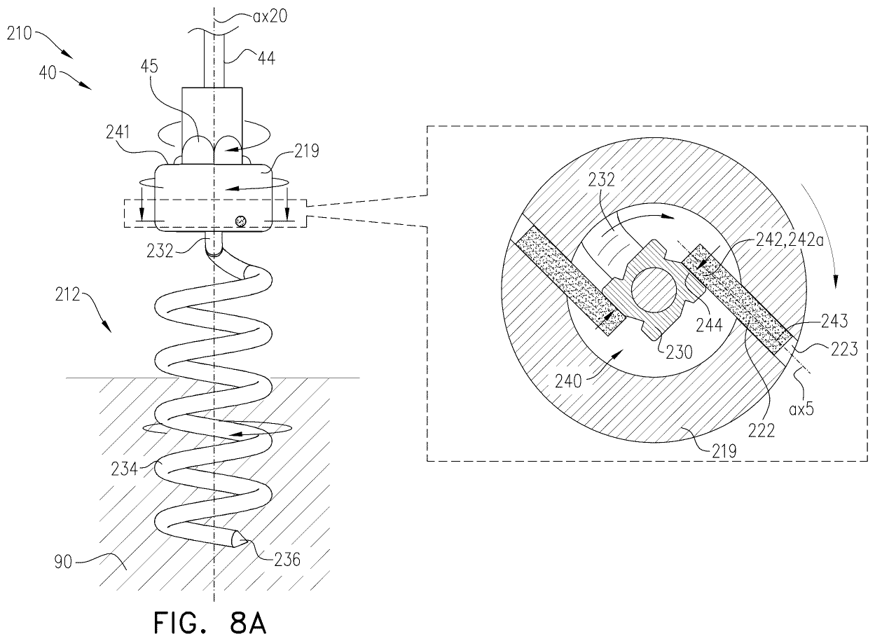

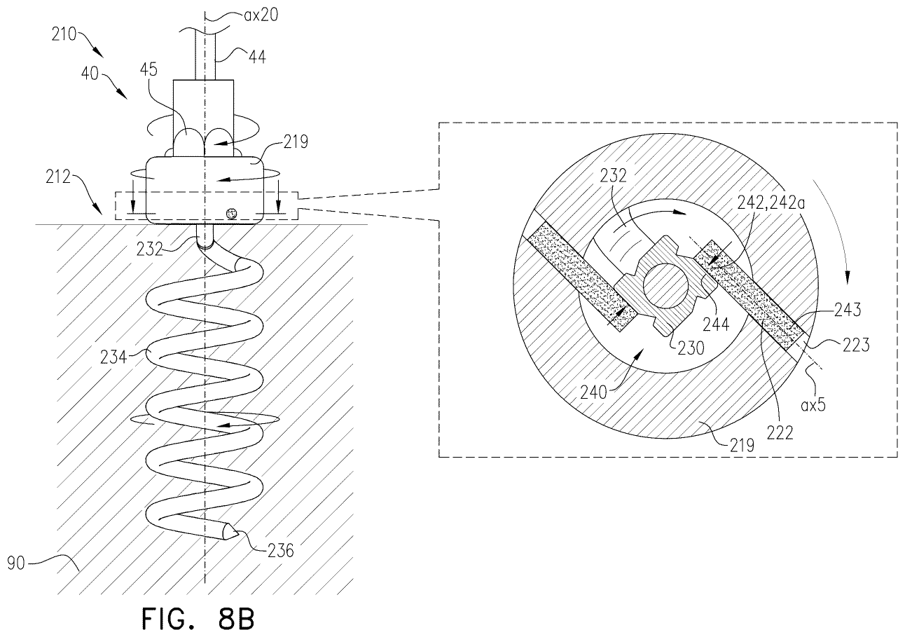

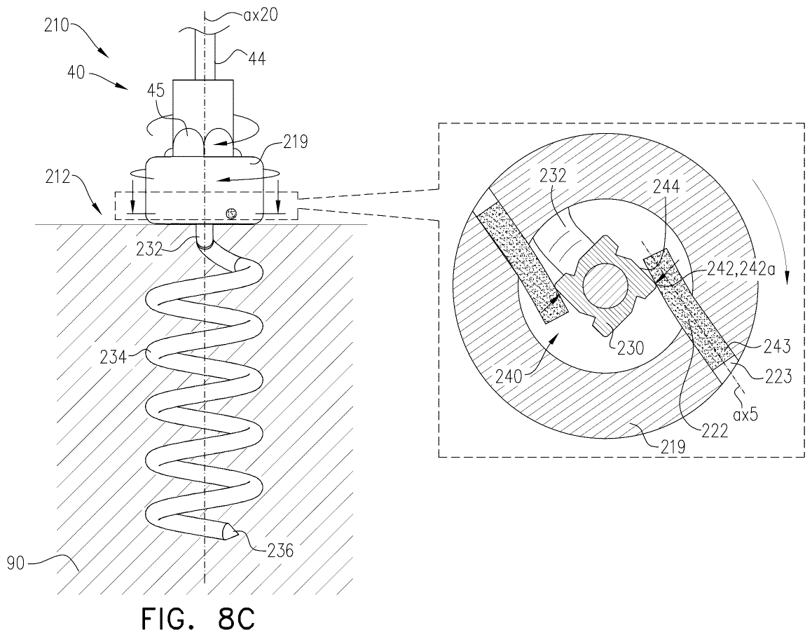

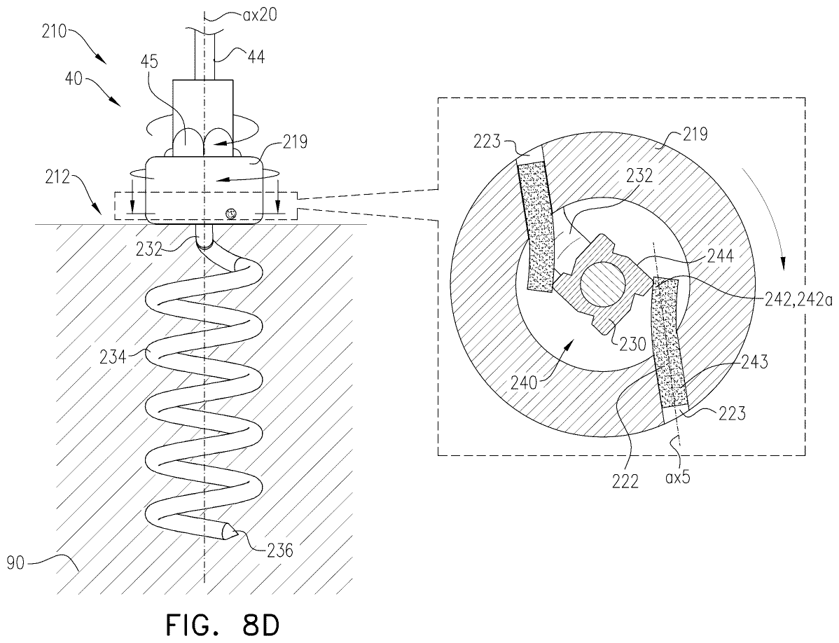

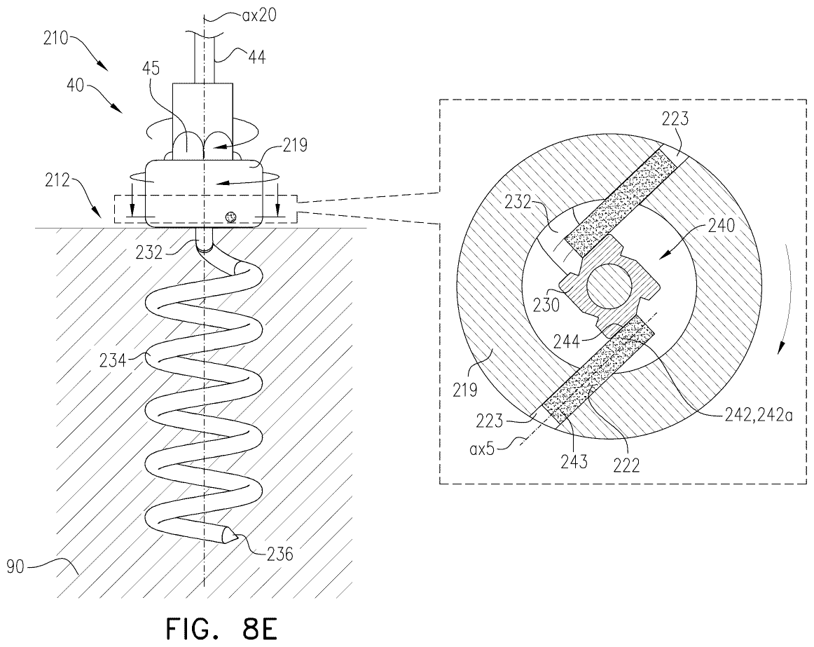

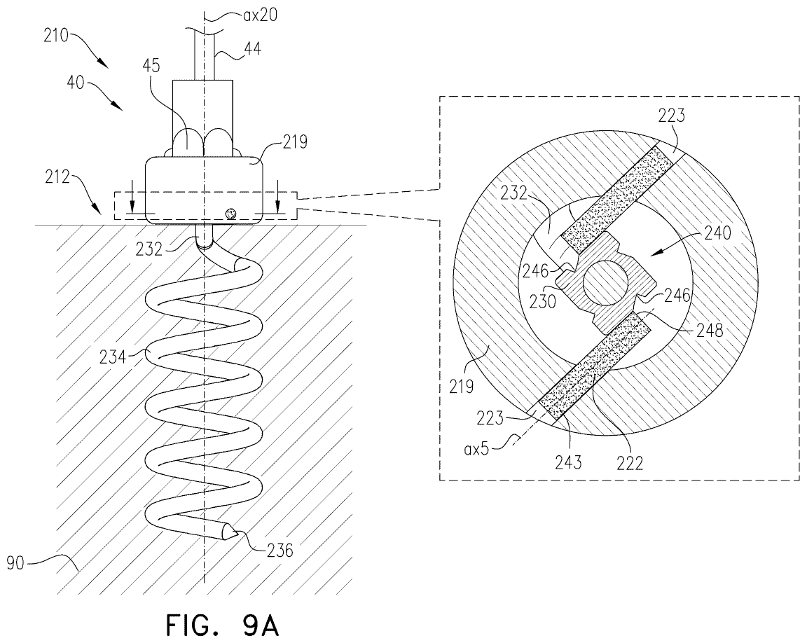

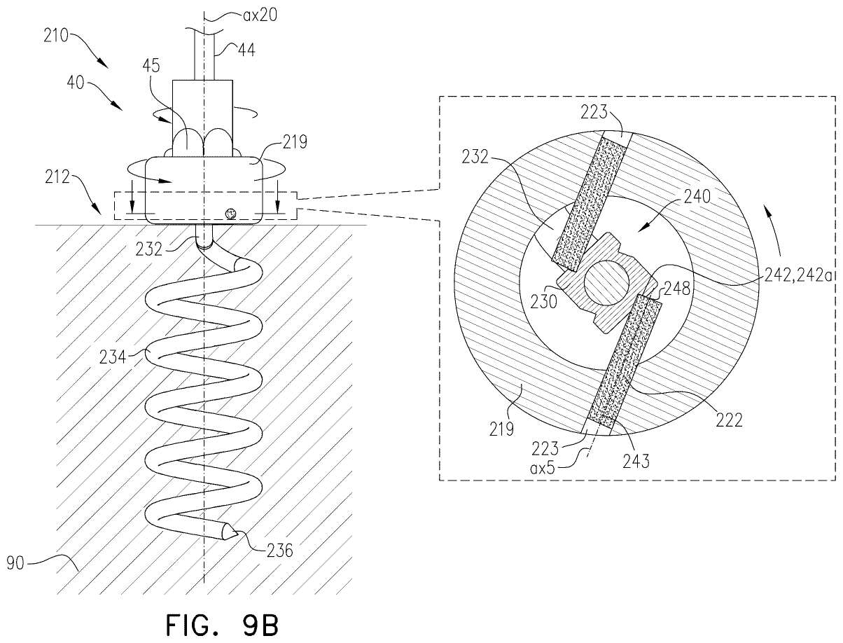

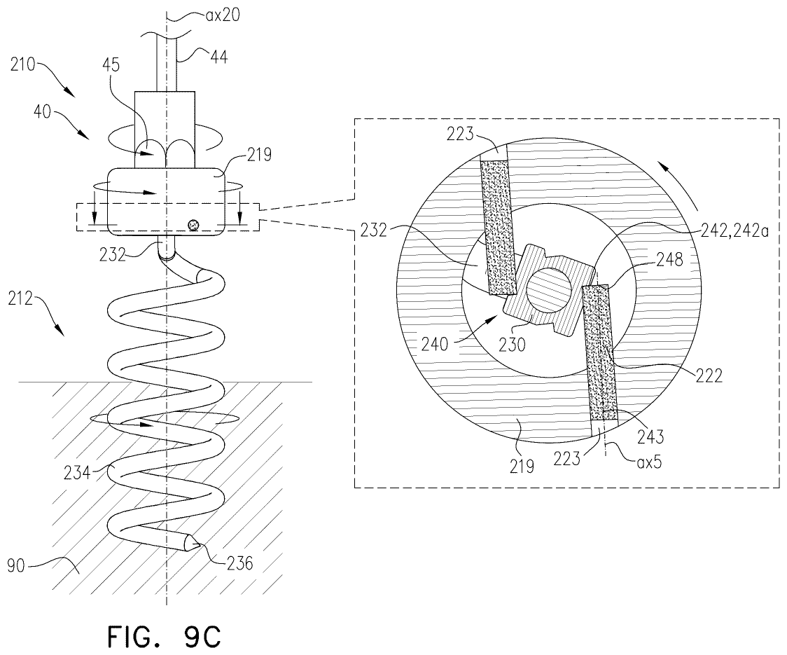

Reference is made to FIGS. 4A-D, 5A-E, and 6A-B, which are schematic illustrations showing an example tissue anchor system 110, in accordance with some applications. Reference is also made to FIGS. 7A-C, 8A-E, and 9A-C, which are schematic illustrations showing an example tissue anchor system 210, in accordance with some applications.

Systems 10, 110 and 210 have several features in common with each other. Furthermore, components that are identically named between the systems typically share similar features and serve similar functions as each other. For example, each of tissue anchors 112 and 212 comprises a driver interface 141, 241 shown being engaged by driver head 45 and rotated using driver 40. As such, the description below of systems 110 and 210 focuses upon features that distinguish each system from system 10.

Systems 110 and 210 are described as comprising anchor driver 40 (described hereinabove in reference to FIGS. 2 and 3A-H), but each of these systems can optionally comprise a different anchor driver.

For some applications, and as shown in FIGS. 4A and 7A, each crown 138, 238 comprises a respective housing 119, 219 which comprises a proximal casing 124, 224 and a distal casing 126, 226. A plurality of grooves 123, 223 (e.g., grooves 123a and 123b, or grooves 223a and 223b, respectively) are shown as being defined by distal casing 126, 226 yet the grooves can optionally be defined by proximal casing 124, mutatis mutandis.

Each crowns 138, 238 respectively comprises an anchor head 130 or gear 230 that is fixedly coupled via a neck 132, 232 to an anchoring portion 134, 234, having a distal tissue-piercing point 136, 236, such that rotation of the anchor head or gear rotates the anchoring portion about a longitudinal axis ax10, ax20, as described hereinabove in reference to anchor 12.

Crowns 138 and 238 of tissue anchors 112 and 212 do not utilize a socket in the manner described for crown 38 of anchor 12. Instead, each of crowns 138 and 238 comprises elements that function together as a slip clutch 140, 240 that couples (e.g., selectively rotatably couples) their respective driver interface 141, 241 to the respective anchor head 130 or gear 230 of crowns 138, 238.

Selective rotational coupling of interface 141, 241 to anchor head 130 or gear 230 by way of slip clutch 140, 240 facilitates transfer of torque from the driver interface to the anchor head, yet limits the transferred torque such that the torque does not exceed a torque threshold. It is hypothesized that using a slip clutch to limit the transferred torque reduces a risk of overtightening the anchor or damaging the tissue. It is further hypothesized that, for some applications, using a slip clutch in this manner may also reduce a risk of under-tightening the anchor, by enabling a surgeon to confidently tighten the anchor without inadvertently overtightening.

As shown in FIGS. 4A-D, slip clutch 140 defined by crown 138 of tissue anchor 112 comprises one or more cantilever pins 122 disposed along a respective groove axis ax5 (e.g., a first cantilever pin 122a disposed along a first groove axis ax5a, and a second cantilever pin 122b disposed along a second groove axis ax5b), such that each pin is disposed within a respective groove 123 defined by housing 119. As shown in FIG. 5A, groove 123 comprises a loose portion 135, within which a free portion 145 of pin 122 is disposed, and a tight portion 133 within which a fixed portion 143 of the pin is disposed (e.g., such that the fixed portion is fixedly coupled to driver interface 141). A loose-portion width d2 of the loose portion can be greater than a tight-portion width d1 of the tight portion.

In some applications, and as shown in FIG. 5A, each groove axis ax5 lies on a groove plane that is generally perpendicular to longitudinal axis ax1 and longitudinally aligned with anchor head 130. In this way, each of pins 122 is longitudinally aligned with anchor head 130. In transverse cross-section (e.g., FIG. 5A), anchor head 130 has a non-circular profile, defining a plurality of lateral surfaces (e.g., sides) 144. Although anchor head 130 is shown as having a square profile, this is not meant to exclude other shapes (e.g., other polygons, such as a hexagon). In a resting state of anchor 112 (e.g., as shown in FIGS. 4B and 4D), each of pins 122 is in contact with a lateral surface 144 of anchor head 130.

In some applications, and as shown, anchor head 130 is coupled to a bearing 120 that is housed within housing 119 such that the bearing is rotationally coupled to the housing, and rotationally couples the anchor head and anchoring portion 134 to the housing. In some applications, bearing 120 is housed snugly within housing 119 so as to provide smooth rotation with little wobble. In this way, rotation of driver interface 141 rotates housing 119, yet whether rotation of the housing will rotate bearing 120, anchor head 130 and anchoring portion 134, is dependent upon contact between cantilever pins 122 (e.g., lateral surface 144 thereof) and the anchor head—i.e., on slip clutch 140.

In some applications, rotational coupling of driver interface 141 to anchor head 130 is accomplished via contact between cantilever pins 122 and the anchor head, e.g., by the cantilever pins pressing against lateral surfaces 144 of the anchor head. For example, and as described in more detail hereinbelow, the system can be configured such that application, to interface 141, of torque below the torque threshold, rotates the housing 119, pins 122 and anchor head 130 in unison, while the pins remain in contact with lateral surfaces 144 of the anchor head. However, application of torque above the torque threshold will typically cause the anchor head to push against the pins, such that the pins deflect laterally away from longitudinal axis ax10, while the pins (and housing 119) revolve around the anchor head. In this way, torque exceeding the torque threshold may not be transferred to anchor head 130.

For some applications, and as shown in FIG. 5A, torque is transferred to lateral surface 144 of anchor head 130, from a portion of pins 122, e.g., from a torque-applying portion 142 (e.g., a first torque-applying portion 142a), between fixed portion 143 and free portion 145—that is in contact with the lateral surface of the anchor head. For some such applications, while screwing anchoring portion 134 into tissue 90, torque-applying portion 142 comprises a leading end of pin 122, such that the torque-applying portion revolves ahead of fixed portion 143 while pin 122 revolves about axis ax10.

For some applications, and as shown, torque-applying portion 142 is defined merely by virtue of being the portion of pin 122 via which torque is applied to anchor head 130, rather than being a physical or other distinguishing feature of that portion of the pin.

The rotational arrows in FIG. 5A indicate that torque applied to driver interface 141, using driver 40, causes housing 119, anchor head 130 and anchoring portion 134 to rotate with pins 122, thereby facilitating screwing the anchor 112 into tissue 90.

FIG. 5B shows anchor 112 having been screwed into tissue 90, due to continued application of torque, to anchor interface 141, in the first direction (e.g., forward torque applied in a forward direction). Screwing anchor 112 into tissue 90 has moved the anchor distally, such that a tissue-facing surface 128 contacts the tissue. At this point, resistance provided by the tissue to further distal movement of tissue-facing surface 128 increases the magnitude of torque required to continue to rotate driver interface 141 above the torque threshold (e.g., increasing the required torque from a first magnitude that is below the torque threshold, to a second magnitude that is above the torque threshold).

As shown in FIG. 5C, anchor head 130 (e.g., lateral surface 144 thereof) begins to deflect pin 122 away from longitudinal axis ax10, such that the pins are not entirely parallel to groove axis ax5, and slip clutch 140 begins to slip.

In some applications, pins 122 are sufficiently flexible to deflect, while torque is applied to interface 141 at above the torque threshold, and torque-applying portion 142a contacts anchor head 130 at one end of a cantilever span (e.g., a forward cantilever span d3), while fixed portion 143 of the pin is fitted within tight portion 133 at another end of the cantilever span. Thus, the forward cantilever span is typically measured along the pin from (i) torque-applying portion 142a, to (ii) fixed portion 143. Flexibility of pin 122 and/or a length of forward cantilever span d3 can be configured in order to set the torque threshold of slip clutch 140.

FIG. 5D shows slip clutch 140 having continued to slip, such that pins 122 have deflected further away from longitudinal axis ax1, and an inter-pin distance d5 between points of contact of respective pins 122 with anchor head has increased, as free portions 145 of the pins pivot within loose portions 135 of grooves 123. At this stage, pins 122 have begun to slip (i.e., revolve) around anchor head 130, such that continued application of torque at the second magnitude causes driver interface 141 to rotate with respect to the anchor head and anchoring portion 134—i.e., to rotate without further screwing of the anchoring portion into the tissue.

As shown in FIG. 5E, further rotation of interface 141 allows pins 122 to deflect medially toward their original conformation, and inter-pin distance d5 is reduced, as the interface and the pins complete a quarter turn since their orientation shown in FIG. 5B.

In certain situations, it may be desirable to remove tissue anchor 112 from tissue 90 (e.g., after having partially or fully screwed the tissue anchor into the tissue). For instance, the surgeon may choose to move the anchor (e.g., to an alternate portion of an implant, and/or to an alternate location of tissue), or to remove the anchor entirely (e.g., due to the anchor no longer being necessary).

As shown in FIGS. 6A-B, removal of anchor 112 from tissue 90 is accomplished by applying reverse torque (i.e., torque in a second rotational direction that is opposite to the first rotational direction) to interface 141, such that pins 122 revolve about longitudinal axis ax10 in the second rotational direction, while the pins contact lateral surface 144 of the anchor head.

For some applications, it may be important to ensure that sufficient reverse torque can be applied to unscrew the anchor, despite the original anchoring torque having been limited. Furthermore, in some cases, the surgeon may encounter greater resistance to unscrewing anchor 121, than that encountered when initially screwing the anchor into tissue 90. For instance, development of scar tissue at an implantation site of anchor 121 may impede removal of the anchor. In order to facilitate unscrewing of anchor 112 from tissue 90, some applications of tissue anchor 112 allow more reverse torque than forward torque to be transferred from driver interface 141 to anchor head 130.

Therefore, for some such applications, reverse torque exceeding the torque threshold (e.g., at the second magnitude) can be transferred from driver interface 141 to anchor head 130. That is, pins 122 are sufficiently rigid to resist deflection while reverse torque is applied at the second magnitude to interface 141, and torque-applying portion 142b contacts anchor head 130 at one end of reverse cantilever span d4, while fixed portion 143 of the pin is fitted within tight portion 133 at another end of the reverse cantilever span. For some such applications, while unscrewing anchoring portion 134 from tissue 90, torque-applying portion 142 comprises a leading end of pin 122, such that the torque-applying portion revolves ahead of fixed portion 143 while the pin revolves about axis ax10.

As shown, the reverse cantilever span is typically measured along the pin from (i) torque-applying portion 142b, to (ii) fixed portion 143. Reverse cantilever span d4 is typically shorter than forward cantilever span d3, such that torque-applying portion 142b is closer to fixed portion 143 when unscrewing anchor 112, than torque-applying portion 142a is to fixed portion 143 when screwing the anchor into tissue 90. It is hypothesized that a magnitude of torque that can be applied, via pin 122, from anchor interface 141 to anchor head 130, is inversely related to the length of the cantilever span, such that a greater magnitude of torque can be transferred along a shorter cantilever span.

However, it may be desirable to limit also the magnitude of reverse torque that can be applied. Therefore, for some applications, slip clutch 140 limits the magnitude of reverse torque that can be applied from interface 141 to anchor head 130 while unscrewing tissue anchor 112. For such applications, a second torque threshold (i.e., a reverse torque threshold), greater than the first torque threshold, is typically established. Thereby, application of torque at a third magnitude, exceeding the second torque threshold, may cause slip clutch 140 to slip, and driver interface 141 to rotate with respect to anchor head 130 and anchoring portion 134.

Reference is made again to FIGS. 7A-C, 8A-E, and 9A-C, which show tissue anchor 212 of system 210. As described hereinabove, tissue anchor 212 shares features with tissue anchor 112. As such, the description hereinbelow focuses upon features that distinguish anchor 212 from anchor 112, particularly features of slip clutch 240 which differ from those of slip clutch 140. For example, slip clutch 240 comprises a gear 230 in place of anchor head 130, and a cantilever pin 222 in place of pin 122. Slip clutch 240 is therefore a ratcheting slip clutch in which pin 222 defines a pawl that interacts with gear 230 as described in greater detail hereinbelow.

Similarly to slip clutch 140, slip clutch 240 facilitates transfer of torque from the driver interface to the anchor head, yet limits the magnitude of torque that can be applied when screwing tissue anchor 212 into tissue 90, by selectively rotationally coupling driver interface 241 to gear 230.

Grooves 223 are dimensioned to snugly fit pins 222, similarly to way that tight portions 133 fit pins 122, and pins 222 are typically dimensioned such that while fixed portions 243 thereof are disposed within grooves 223, the pins (e.g., torque-applying portions 242 thereof) are in contact with gear 230 (e.g., a non-circular lateral surface 244 thereof). In a resting state of anchor 212 (e.g., as shown in FIGS. 7B and 7C), each of pins 222 is in contact with lateral surface 244 of gear 230.

For some applications, and similarly to as described hereinabove in reference to anchor head 130 of system 110, gear 230 is coupled to a bearing 220 that is housed within housing 219 such that the bearing is rotationally coupled to the housing, and rotationally couples the gear and anchoring portion 234 to the housing. For some applications, bearing 220 is housed snugly within housing 219 so as to provide smooth rotation with little wobble. In this way, rotation of driver interface 241 rotates housing 219, yet whether rotation of the housing will rotate bearing 220, gear 230 and anchoring portion 234, is dependent upon contact between pins 222 (e.g., lateral surface 244 thereof) and the gear.

FIG. 8A shows anchor 212 being screwed into tissue 90. The rotational arrows in FIG. 8A indicate that torque applied to driver interface 241, using driver 40, causes housing 219, gear 230 and anchoring portion 234 to rotate, thereby screwing anchoring portion 234 into tissue 90. Similarly to as described hereinabove in reference to slip clutch 140, while torque is applied to interface 241 at under the torque threshold, pins 222 revolve about longitudinal axis ax20 while torque-applying portions 242a of the pins are in contact with lateral surface 244 of gear 230, causing the gear and anchoring portion 234 to rotate. However, in contrast that described hereinabove with reference to slip clutch 140, while screwing anchoring portion 234 into tissue 90, fixed portion 243 comprises a leading end of pin 222, such that the fixed portion revolves ahead of torque-applying portion 242 while the pin revolves about axis ax20.

FIG. 8B shows anchor 212 having been screwed into tissue 90, due to continued application of forward torque to anchor interface 241. Screwing anchor 212 into tissue 90 has moved the anchor distally, such that a tissue-facing surface 228 contacts the tissue. As described hereinabove in reference to tissue anchor 121, resistance provided by the tissue to further distal movement of tissue-facing surface 228 increases the magnitude of torque required to rotate driver interface 241 (e.g., from the first magnitude to the second magnitude) to beyond the torque threshold.

As shown in FIG. 8C, gear 230 (e.g., lateral surface 244 thereof) begins to deflect torque-applying portions 242a of pins 222 laterally away from longitudinal axis ax20, such that the pins are not entirely parallel to axis ax5. Pins 222 begin to slip around gear 230, thereby reducing the torque transferred from driver interface 241 to gear 230. FIG. 8D shows gear 230 having further deflected pins 222 (e.g., torque-applying portions 242a thereof) away from longitudinal axis ax20, such that slip clutch 240 has continued to slip around gear 230. At this stage, application of forward torque at the second magnitude causes driver interface 241 and pins 222 to rotate with respect to gear 230 and anchoring portion 234.

As shown in FIG. 8E, further rotation of interface 241 allows pins 222 to deflect medially toward their original conformation, as the interface and the pins complete a quarter turn since their orientation shown in FIG. 8B.

Similarly to as described hereinabove regarding tissue anchor 112 with reference to FIGS. 6A-B, it may be desirable in certain situations to remove tissue anchor 212 from tissue 90 (e.g., after having partially or fully screwed the tissue anchor into the tissue).

As shown in FIGS. 9A-B, removal of anchor 212 from tissue 90 is accomplished by applying reverse torque to interface 241, such that pins 222 revolve about longitudinal axis ax20 in the second rotational direction, while the pins contact gear 230.

However, the manner in which pins 222 of anchor 212 contact gear 230 while revolving about longitudinal axis ax20 in the second rotational direction is different from the manner in which the pins contact the gear while revolving in the first direction. As shown in FIG. 9B, reverse rotation of driver interface 241 can cause some reverse rotation of pins 222 in relation to gear 230 (e.g., “backlash”). As shown, a degree of backlash permitted by reverse rotation of pins 222 is typically limited to less than a quarter turn.

FIG. 9B shows pins 222 having rotated in the second direction until the pins engage gear 230 (e.g., until end-portions 248 are latched into notches 246). In some applications, once end-portions 248 fit into notches 246, backlash is stopped, and pins (e.g., torque-applying portions 242b) transfer torque from interface 241 to gear 230, this time in the second direction.

Thereby, slip clutch 240 may allow reverse torque that exceeds the torque threshold to be transferred from driver interface 241 to gear 230. As shown in FIGS. 9B-C, latching of end-portions 248 of pins 222 into notches 246 facilitates unscrewing anchor 212 from tissue 90, by gear 230 and anchoring portion 234 rotating with driver interface 241, in response to pin 222 revolving about longitudinal axis ax20 in the second direction.

Referring again to FIGS. 1A-9C, the tissue anchors described herein can be used to fasten one tissue to another, and/or to secure another element (e.g., an implant) to tissue. For example, a system for treating a patient can include an implant that is secured to the tissue with any of the tissue anchors described herein.

The tissue anchors described herein can be used for anchoring to cardiac tissue, such as to an atrial wall, a ventricular wall, or a valve annulus of a heart. For some applications, one or more of the tissue anchors described herein can be used to secure, to cardiac tissue, an implant. For some applications, one or more of the tissue anchors described herein can be used to secure, to cardiac tissue, a tether or contraction member (e.g., contraction wire, contraction ribbon, contraction suture, etc.) that is to be tensioned in order to change a shape and/or function of the heart. For example, one or more of the tissue anchors described herein can be used to secure, to ventricular tissue, a tether or contraction member that is also secured to a valve leaflet. For some applications, a plurality of tissue anchors described herein can be used to secure, to a valve annulus, an annuloplasty structure, e.g., with the anchors serving, mutatis mutandis, in place of the anchors described in one or more of the embodiments disclosed in U.S. Pat. No. 9,949,828 to Sheps et al., US Patent Application Publication 2020/0015971 to Brauon et al., PCT Application PCT/IB2020/060044 to Kasher et al. (which published as WO 2021/084407), and/or U.S. Provisional Patent Application 63/147,699 to Shafigh et al., each of which is incorporated herein by reference. Furthermore, one or more features of the tissue anchors described in these incorporated references cab be provided on any of the tissue anchors described hereinabove. For example, the scope of the present disclosure includes modifying any of the anchors described hereinabove to include (e.g., on the crown of the tissue anchor) an eyelet or rotating eyelet, such as described in one of these incorporated references, e.g., in order to slidably couple the anchor to a tether or contraction member that is to be anchored around an annulus of a heart valve and subsequently tensioned in order to perform transluminal annuloplasty. For some applications, an implant comprises a tether/contraction member and one or more of the anchors described herein.

The present invention is not limited to what has been particularly shown and described herein. Rather, the scope of the invention includes both combinations and subcombinations of the various features described herein, as well as variations and modifications thereof that are not in the prior art, which would occur to persons skilled in the art upon reading the foregoing description. The treatment techniques, methods, operations, steps, etc. described or suggested herein can be performed on a living animal or on a non-living simulation, such as on a cadaver, cadaver heart, simulator (e.g., with the body parts, tissue, etc. being simulated), etc.

We claim:

1. A method for use with a tissue of a subject, the method comprising:
using a driver, advancing a tissue anchor to the tissue, the tissue anchor including:
an anchoring portion,
an anchor head fixedly coupled to the anchoring portion, and
a driver interface;
screwing the anchoring portion distally into the tissue by, while the driver is engaged with the driver interface, applying torque, via the driver, to the driver interface such that the anchor head and the anchoring portion rotate together about a longitudinal axis of the tissue anchor;
continuing to screw the anchoring portion distally into the tissue by continuing to apply the torque at least until the driver interface becomes rotatable relative to the anchor head; and
subsequently, disengaging the driver from the driver interface and removing the driver from the subject, while leaving the tissue anchor anchored to the tissue.

2. The method according to claim 1, wherein:
the tissue anchor further includes a socket,
screwing the anchoring portion distally into the tissue comprises screwing the anchoring portion distally into the tissue while the anchor head is seated snugly within the socket, and
continuing to apply the torque comprises continuing to apply the torque at least until the anchor head exits the socket distally in a manner that makes the driver interface rotatable relative to the anchor head.

3. The method according to claim 2, wherein continuing to screw the anchoring portion distally into the tissue comprises continuing to screw the anchoring portion distally into the tissue until the tissue pulls the anchor head distally out of the socket.

4. The method according to claim 2, wherein:
the driver interface defines a floor, and
screwing the anchoring portion distally into the tissue comprises screwing the anchoring portion distally into the tissue while the floor separates the driver from the anchor head and the driver interface is engaged by the driver.

5. The method according to claim 2, wherein:
the tissue anchor includes a crown coupled to a proximal portion of the anchoring portion, the crown defining the socket and a tissue-facing surface facing distally away from the socket, and
continuing to screw the anchoring portion distally into the tissue comprises continuing to screw the anchoring portion distally into the tissue until the tissue-facing surface presses against the tissue.

6. The method according to claim 5, wherein:
the crown includes a casing, the casing dimensioned to define:
the driver interface,
the socket,
the tissue-facing surface, and
a free zone disposed between the socket and the tissue-facing surface; and
continuing to screw the anchoring portion distally into the tissue comprises continuing to screw the anchoring portion distally into the tissue at least until the anchor head becomes pulled distally into the free zone.

7. The method according to claim 6, wherein:
the tissue anchor further includes a spring disposed within the casing, between the anchor head and the tissue-facing surface, and
continuing to screw the anchoring portion distally into the tissue comprises continuing to screw the anchoring portion distally into the tissue until the tissue pulls the anchor head distally out of the socket, thereby compressing the spring.

8. The method according to claim 1, wherein the steps of (i) screwing the anchoring portion and (ii) continuing to screw the anchoring portion are steps of securing an implant to the tissue using the tissue anchor.

9. The method according to claim 8, wherein:
the implant includes a tether, and
securing the implant to the tissue comprises securing the tether to the tissue such that applying tension to the tether changes a shape of the tissue.

10. The method according to claim 1, wherein:
the tissue anchor further includes a slip clutch, coupled to the anchor head,
screwing the anchoring portion distally into the tissue comprises screwing the anchoring portion distally into the tissue while the slip clutch transfers the torque to the anchor head, and
continuing to screw the anchoring portion distally into the tissue comprises continuing to screw the anchoring portion distally into the tissue until the slip clutch slips in a manner that allows the driver interface to rotate relative to the anchor head.

11. The method according to claim 10, wherein:
the anchor head is shaped to define a non-circular lateral surface,
the slip clutch includes a cantilever pin,
screwing the anchoring portion distally into the tissue comprises screwing the anchoring portion distally into the tissue while a torque-applying portion of the pin transfers torque to the non-circular lateral surface of the anchor head, and
continuing to screw the anchoring portion distally into the tissue comprises continuing to screw the anchoring portion distally into the tissue until the slip clutch slips via the pin deflecting with respect to the anchor head.

12. The method according to claim 11, wherein the pin has a fixed portion that is fixedly coupled to the driver interface, and wherein screwing the anchoring portion distally into the tissue comprises screwing the anchoring portion distally into the tissue by applying the torque to the driver interface such that it causes the cantilever pin to revolve about the longitudinal axis with the torque-applying portion ahead of the fixed portion.

13. The method according to claim 11, wherein the pin has a fixed portion that is fixedly coupled to the driver interface, and wherein screwing the anchoring portion distally into the tissue comprises screwing the anchoring portion distally into the tissue by applying the torque to the driver interface such that it causes the cantilever pin to revolve about the longitudinal axis with the torque-applying portion trailing the fixed portion.

14. The method according to claim 10, wherein:
the torque is forward torque,
screwing the anchoring portion distally into the tissue comprises screwing the anchoring portion distally into the tissue by applying the forward torque at a first magnitude that does not exceed a torque threshold defined by the slip clutch,
continuing to screw the anchoring portion distally into the tissue comprises continuing to screw the anchoring portion distally into the tissue by continuing to apply the forward torque until the forward torque exceeds the torque threshold, responsively to which the slip clutch slips, and
the method further comprises unscrewing the anchoring portion proximally out of the tissue by, while the driver remains engaged with the driver interface, applying, to the driver interface, reverse torque at a second magnitude that exceeds the torque threshold.

15. The method according to claim 14, wherein:
the anchor head is shaped to define a non-circular lateral surface,
the slip clutch includes a cantilever pin, a fixed portion of the cantilever pin fixedly coupled to the driver interface, and
screwing the anchoring portion distally into the tissue comprises screwing the anchoring portion distally into the tissue while a torque-applying portion of the pin is in contact with the non-circular lateral surface of the anchor head, and
continuing to screw the anchoring portion distally into the tissue comprises continuing to screw the anchoring portion distally into the tissue by continuing to apply the forward torque until the slip clutch slips via the pin being deflected away from the longitudinal axis by the anchor head.

16. The method according to claim 15, wherein:
the torque-applying portion is a first torque-applying portion,
screwing the anchoring portion distally into the tissue comprises screwing the anchoring portion distally into the tissue by rotating the driver interface such that the first torque-applying portion transfers the forward torque to the non-circular lateral surface of the anchor head, and
unscrewing the anchoring portion proximally out of the tissue comprises unscrewing the anchoring portion proximally out of the tissue by rotating the driver interface such that a second torque-applying portion of the pin transfers the reverse torque to the anchor head.

17. The method according to claim 16, wherein:
the anchor head is shaped to define a notch,
the pin defines a pawl, which serves as the second torque-applying portion, and
unscrewing the anchoring portion proximally out of the tissue comprises unscrewing the anchoring portion proximally out of the tissue by applying the reverse torque to the driver interface such the pawl transfers the reverse torque to the anchor head by latching into the notch.

18. The method according to claim 17, wherein applying the reverse torque to the driver interface comprises applying the reverse torque to the driver interface such that the driver interface rotates for up to a quarter turn before the pawl latches into the notch and transfers the reverse torque to the anchor head.
